(19) United States
(12) Patent Application Publication
(10) Pub. No.: US 2026/0108220 A1
(43) Pub. Date: Apr. 23, 2026

(54) SCAN SUBJECT SUPPORT APPARATUS, MEDICAL IMAGING SCANNING SYSTEM, AND ANCHORING CONTROL METHOD

(71) Applicant: GE Precision Healthcare LLC, Waukesha, WI (US)

(72) Inventors: Yigang Xia, Beijing (CN); Yifei Dong, Beijing (CN); Qian Xu, Beijing (CN)

(21) Appl. No.: 19/364,859

(22) Filed: Oct. 21, 2025

(30) Foreign Application Priority Data
Oct. 21, 2024 (CN) 202411471406.8

(51) Int. Cl. A61B 6/04; A61B 6/03

(52) U.S. Cl. CPC A61B 6/0407; A61B 6/032

(57) ABSTRACT

Examples of a scan subject support apparatus, a medical imaging scanning system, and an anchoring control method are described. The support apparatus includes a support platform for a scan subject, a lifting drive portion for the support platform, and a base for the lifting drive portion. The support apparatus further includes a moving portion mounted on the base and capable of moving the support apparatus, and an electromagnetic locking apparatus, including a first anchoring portion disposed on the base and a second anchoring portion disposed at a position-to-be-fixed of the support apparatus. The electromagnetic locking apparatus engages the first anchoring portion with the second anchoring portion by using an electromagnetic force when being turned on, to fix the support apparatus at the position-to-be-fixed.

### CROSS-REFERENCE TO RELATED APPLICATIONS

This application claims priority to Chinese Application No. 202411471406.8, filed on Oct. 21, 2024, the disclosure of which is incorporated herein by reference in its entirety.

### TECHNICAL FIELD

Examples of the present application relate to the technical field of medical devices, and in particular to a scan subject support apparatus, a medical imaging scanning system, and an anchoring control method.

### BACKGROUND

A scan subject support apparatus is an important auxiliary device for a medical imaging scanning system, and is required to lift and lower a scan subject and transport the scan subject to a scanning position in a scanning apparatus. The scan subject support apparatus includes a support platform. During scanning, the support platform may move toward the scanning apparatus (a movement direction may be referred to as a moving-out direction) to move the scan subject to a suitable scanning position. After scanning is completed, the support platform may move in a direction opposite to the moving-out direction to bring the scan subject back to an original position.

Usually, the scan subject (for example, a patient) needs to lie on the support platform or leave the support platform during scanning. However, an additional measure (for example, a stretcher) is required to assist a scan subject with limited mobility (for example, a critically ill, paralyzed, or fracture patient) in getting on and off the support platform. Such a measure may cause harm to the scan subject. In another measure, a movable scan subject support apparatus or a movable scanning apparatus may be used to move the two together during imaging scanning.

It should be noted that the above introduction of the background is only for the convenience of clearly and completely describing the technical solutions of the present application, and for the convenience of understanding for those skilled in the art.

### SUMMARY OF THE INVENTION

During scanning, a support platform of a movable scan subject support apparatus bears a scan subject and moves and extends into a scanning apparatus. The weight of the support platform and the scan subject deviates from a main body of the scan subject support apparatus, that is, the center of gravity of the entire scan subject support apparatus changes. In this case, it is difficult to balance a load of the scan subject, that is, a side of the scan subject support apparatus close to the scanning apparatus may tilt. Consequently, stability of the scan subject support apparatus cannot be effectively maintained, and imaging quality of imaging scanning is further affected. On the other hand, if the scan subject is accidentally moved or contacted or the scan subject support apparatus is accidentally collided with during scanning, the scan subject support apparatus may move, and the scan subject support apparatus needs to be re-positioned or re-aligned with the scanning apparatus, which affects efficiency or imaging quality of an imaging scanning operation.

For at least one of the foregoing technical problems or another similar problem, examples of the present application provide a scan subject support apparatus, a medical imaging scanning system, and an anchoring control method. Even if a movable scan subject support apparatus is used, a load of a scan subject can be easily balanced, thereby effectively maintaining stability of the scan subject support apparatus.

According to an aspect of the examples of the present application, provided is a scan subject support apparatus, including a support platform for bearing a scan subject, a lifting drive portion for bearing the support platform, and a base for bearing the lifting drive portion. The scan subject support apparatus includes a moving portion, mounted on the base and capable of moving the entire scan subject support apparatus; and an electromagnetic locking apparatus, including a first anchoring portion disposed on the base and a second anchoring portion disposed at a position-to-be-fixed of the scan subject support apparatus, wherein the electromagnetic locking apparatus engages the first anchoring portion with the second anchoring portion by using an electromagnetic force when being turned on, to fix the scan subject support apparatus at the position-to-be-fixed.

In some examples, the moving portion includes a plurality of casters disposed on the lower side of the base; the plurality of casters include a first caster located on a side of the scan subject support apparatus close to a scanning apparatus during scanning and a second caster located on a side away from the scanning apparatus; and the first anchoring portion is disposed at an end of the base away from the scanning apparatus, and is located on a same side of the base as the second caster. In some examples, the first anchoring portion includes a magnetically conductive fixture, the second anchoring portion disposed at the position-to-be-fixed includes an electromagnet, and the electromagnetic force is generated by the magnetically conductive fixture and the electromagnet; or the first anchoring portion includes an electromagnet, the second anchoring portion disposed at the position-to-be-fixed includes a magnetically conductive fixture, and the electromagnetic force is generated by the magnetically conductive fixture and the electromagnet.

In some examples, the scan subject support apparatus further includes a pressing apparatus, located on the lower side of the scan subject support apparatus, connected to the first anchoring portion by using a connection module, and capable of moving the first anchoring portion toward the second anchoring portion. In some examples, the pressing apparatus includes a pedal, moving the first anchoring portion toward the second anchoring portion by using a rotating shaft; and a guide piece, guiding the first anchoring portion, to limit a direction in which the first anchoring portion moves toward the second anchoring portion. In some examples, the pressing apparatus further includes a restoration piece, restoring the pressing apparatus in a pressed state to an initial position when the electromagnetic locking apparatus is turned off.

In some examples, the second anchoring portion is disposed on the ground, and a positioning hole is further disposed at the position-to-be-fixed; the pressing apparatus further includes a positioning piece protruding downward from the first anchoring portion; the positioning piece is inserted into the positioning hole when the pressing apparatus is pressed to move the first anchoring portion toward the second anchoring portion; and the positioning piece is separated from the positioning hole when the pressing apparatus is restored to a non-pressed state.

In some examples, the scan subject support apparatus further includes a handle portion, located on a side of the scan subject support apparatus away from a scanning apparatus during scanning, and capable of controlling the scan subject support apparatus to move. In some examples, the handle portion includes a magnetic switch, turning on or off the electromagnetic locking apparatus by turning on or off a power supply. In some examples, the scan subject support apparatus further includes a connection and fixation apparatus, capable of fixing the scan subject support apparatus to a scanning apparatus.

According to another aspect of the examples of the present application, provided is a medical imaging scanning system. The system includes a scanning apparatus and the foregoing scan subject support apparatus.

According to still another aspect of the examples of the present application, provided is an anchoring control method. The method is applied to a movable scan subject support apparatus. The scan subject support apparatus includes a support platform for bearing a scan subject, a lifting drive portion for bearing the support platform, and a base for bearing the lifting drive portion, and the scan subject support apparatus further includes a moving portion and an electromagnetic locking apparatus. The method includes moving the scan subject support apparatus to a predetermined position, wherein the electromagnetic locking apparatus includes a first anchoring portion disposed on the base and a second anchoring portion disposed at a position-to-be-fixed of the scan subject support apparatus, and turning on the electromagnetic locking apparatus and engaging the first anchoring portion with the second anchoring portion by using an electromagnetic force, to fix the scan subject support apparatus at the position-to-be-fixed.

In some examples, after the moving the scan subject support apparatus to a predetermined position, the method further includes fixing, by using a connection and fixation apparatus, the scan subject support apparatus to a scanning apparatus. In some examples, the method further includes: turning on, by using a magnetic switch of the scan subject support apparatus, a power supply to turn on the electromagnetic locking apparatus. In some examples, the method further includes: shortening, by using a pressing apparatus of the scan subject support apparatus, a distance between the first anchoring portion and the second scanning portion, wherein the first anchoring portion is guided by using a guide piece, and the first anchoring portion is positioned by using a positioning piece and a positioning hole. In some examples, the method further includes: turning off, by using the magnetic switch of the scan subject support apparatus, the power supply to turn off the electromagnetic locking apparatus; and restoring, by using a restoration piece of the pressing apparatus, the pressing apparatus to an initial position.

Beneficial effects of the examples of the present application are as follows: the scan subject support apparatus includes the moving portion and the electromagnetic locking apparatus. The electromagnetic locking apparatus engages the first anchoring portion with the second anchoring portion by using the electromagnetic force when being turned on, to fix the scan subject support apparatus at the position-to-be-fixed. Therefore, a load after at least a part of the scan subject is removed from the support platform can be balanced, thereby improving stability of the support apparatus, and further improving imaging quality of imaging scanning.

On the other hand, the scan subject support apparatus is prevented from moving because the scan subject is accidentally moved or contacted or the scan subject support apparatus is accidentally collided with during scanning, and the scan subject support apparatus and the scanning apparatus are prevented from being re-positioned or re-aligned, thereby improving efficiency or imaging quality of an imaging scanning operation.

With reference to the following description and drawings, specific implementations of the examples of the present application are disclosed in detail, and the way in which the principles of the examples of the present application can be employed are illustrated. It should be understood that the implementations of the present application are not limited in scope thereby. Within the scope of the spirit and clauses of the appended claims, the implementations of the present application include many changes, modifications, and equivalents.

The features described and/or illustrated for one implementation may be used in one or more other implementations in the same or similar way, be combined with features in other implementations, or replace features in other implementations.

It should be emphasized that the terms “include/comprise” when used herein refer to the presence of features, integrated components, steps, or assemblies, but do not preclude the presence or addition of one or more other features, integrated components, steps, or assemblies.

### BRIEF DESCRIPTION OF THE DRAWINGS

The included drawings are used to provide further understanding of the examples of the present application, which constitute a part of the description and are used to illustrate the implementations of the present application and explain the principles of the present application together with textual description. Evidently, the drawings in the following description are merely some examples of the present application, and those of ordinary skill in the art may obtain other implementations according to the drawings without involving inventive effort. In the drawings:

FIG. 1 is a schematic diagram of a CT device according to an example of the present application;

FIG. 2 is a schematic diagram of a CT imaging system according to an example of the present application;

FIG. 3 is a schematic diagram of composition of a medical imaging scanning system according to an example of the present application;

FIG. 4 is a schematic diagram of a scan subject support apparatus according to an example of the present application;

FIG. 5 is a schematic diagram of a movable support portion moving-out platform according to an example of the present application;

FIG. 6 is a schematic diagram of a local part of a scan subject support apparatus according to an example of the present application;

FIG. 7 is a schematic diagram of contact between a scan subject and a scan subject support platform according to an example of the present application;

FIG. 8 is a schematic diagram of a handle portion according to an example of the present application;

FIG. 9 is a schematic diagram of a magnetic switch according to an example of the present application; and

FIG. 10 is a schematic diagram of an anchoring control method according to an example of the present application.

### DETAILED DESCRIPTION

The aforementioned and other features of the examples of the present application will become apparent from the following description with reference to the drawings. In the description and drawings, specific implementations of the present application are disclosed in detail, and part of the implementations in which the principles of the examples of the present application may be employed are indicated. It should be understood that the present application is not limited to the described implementations. On the contrary, the examples of the present application include all modifications, variations, and equivalents which fall within the scope of the appended claims.

In the examples of the present application, the terms “first” and “second” etc., are used to distinguish different elements, but do not represent a spatial arrangement or temporal order, etc., of these elements, and these elements should not be limited by these terms. The term “and/or” includes any and all combinations of one or more associated listed terms. The terms “comprise”, “include”, “have”, etc., refer to the presence of described features, elements, components, or assemblies, but do not exclude the presence or addition of one or more other features, elements, components, or assemblies.

In the examples of the present application, the singular forms “a” and “the”, etc., include plural forms, and should be broadly construed as “a type of” or “a class of” rather than being limited to the meaning of “one”. Furthermore, the term “the” should be construed as including both the singular and plural forms, unless otherwise specified in the context. In addition, the term “according to” should be construed as “at least in part according to . . . ” and the term “on the basis of” should be construed as “at least in part on the basis of . . . ”, unless otherwise specified in the context.

The features described and/or illustrated for one implementation may be used in one or more other implementations in the same or similar way, be combined with features in other implementations, or replace features in other implementations. The term “include/comprise” when used herein refers to the presence of features, integrated components, steps, or assemblies, but does not preclude the presence or addition of one or more other features, integrated components, steps, or assemblies.

The scanning apparatus (that is, a medical imaging scanning system or an imaging scanning apparatus) described in the present application may be suitable for a variety of medical imaging modalities, including but not limited to: computed tomography (CT, Computed Tomography), magnetic resonance imaging (MRI, Magnetic Resonance Imaging), C-arm imaging, positron emission computed tomography (PET, Positron Emission Computed Tomography), single photon emission computed tomography (SPECT, Single Photon Emission Computed Tomography), or any other suitable medical imaging.

For example, the examples of the present application are described below in conjunction with an X-ray computed tomography (CT) imaging system. Those skilled in the art will understand that the examples of the present application can also be applicable to other medical imaging scanning systems.

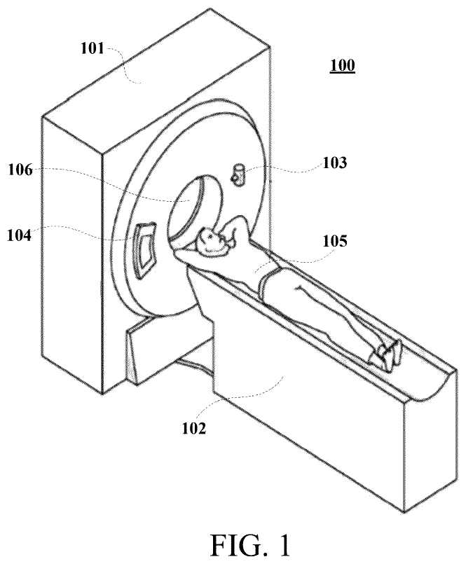

FIG. 1 is a schematic diagram of a CT device according to an example of the present application, and schematically shows a CT device 100. As shown in FIG. 1, the CT device 100 includes a scanning gantry 101 and a patient table 102 (for example, a scanning table). The scanning gantry 101 has an X-ray source 103, and the X-ray source 103 projects an X-ray beam toward a collimator or detector assembly 104 on the opposite side of the scanning gantry 101. An examination subject (also referred to as a scan subject) 105 may lie on the patient table 102 and be moved into a scanning gantry opening 106 along with the patient table 102. Medical imaging data of the examination subject 105 can be obtained through scanning performed by the X-ray source 103.

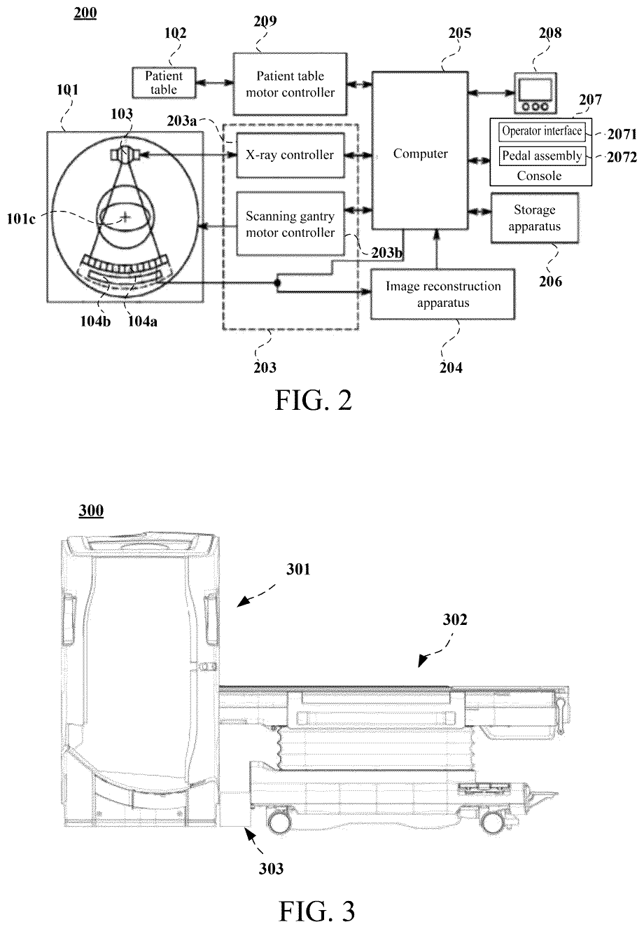

FIG. 2 is a schematic diagram of a CT imaging system according to an example of the present application, and schematically shows a block diagram of a CT imaging system 200. As shown in FIG. 2, the detector assembly 104 includes a plurality of detector units 104a and a data acquisition system (DAS) 104b. The plurality of detector units 104a sense a projected X-ray passing through the examination subject 105.

The DAS 104b, based on sensing of the detector units 104a, converts collected information into projection data for subsequent processing. During the scanning for acquiring the X-ray projection data, the scanning gantry 101 and components mounted thereon rotate around a center of rotation 101c.

Rotation of the scanning gantry 101 and an operation of the X-ray source 103 are controlled by a control mechanism 203 of the CT imaging system 200. The control mechanism 203 includes an X-ray controller 203a that provides power and a timing signal to the X-ray source 103 and a scanning gantry motor controller 203b that controls a rotational speed and a position of the scanning gantry 101. An image reconstruction apparatus 204 receives the projection data from the DAS 104b and performs image reconstruction. A reconstructed image is transmitted as an input to a computer 205, and the computer 205 stores the image in a mass storage apparatus 206.

The computer 205 also receives commands and scanning parameters from an operator through a console 207. The console 207 has an operator interface 2071 in a certain form, such as a keyboard, a mouse, or a speech activated controller. The console 207 may also have an input apparatus such as a pedal assembly 2072. In addition, the console 207 may have another suitable input apparatus. An associated display 208 allows the operator to observe the reconstructed image and other data from the computer 205. The commands and parameters provided by the operator are used by the computer 205 to provide control signals and information to the DAS 104b, the X-ray controller 203a, and the scanning gantry motor controller 203b. In addition, the computer 205 operates a patient table motor controller 209, which controls the patient table 102 to position the examination subject 105 and the scanning gantry 101. In particular, the patient table 102 moves the examination subject 105 to completely or partially pass through the scanning gantry opening 106 in FIG. 1.

The device and system for acquiring medical image data (which may also be referred to as medical images or medical image data) according to the examples of the present application are schematically described above, but the present application is not limited thereto. The medical imaging device may be a CT device, a PET-CT, or any other suitable imaging device. A storage device may be located within the medical imaging device, in a server outside the medical imaging device, in an independent medical imaging storage system (such as a Picture Archiving and Communication System (PACS)), and/or in a remote cloud storage system.

In addition, a medical imaging workstation may be provided locally to the medical imaging device, that is, the medical imaging workstation is provided close to the medical imaging device, and the two may both be located in a scanning room, an imaging department, or the same hospital. In contrast, a medical image cloud platform analysis system may be positioned distant from the medical imaging device, e.g., arranged at a cloud end that is in communication with the medical imaging device.

As an example, after a medical institution completes an imaging scan using the medical imaging device, data obtained by scanning is stored in a storage device. A medical imaging workstation may directly read the data obtained by scanning and perform image processing by means of a processor thereof. As another example, the medical image cloud platform analysis system may read a medical image in the storage device by means of remote communication to provide “software as a service (SaaS)”. SaaS can exist between hospitals, between a hospital and an imaging center, or between a hospital and a third-party online diagnosis and treatment service provider.

Medical imaging scanning is schematically described above, and the examples of the present application are described in detail below with reference to the drawings. In the examples described below, a medical imaging scanning device being a CT device is taken as an example for description, and the content of the description is also applicable to other medical devices.

FIG. 3 is a schematic diagram of composition of a medical imaging scanning system according to an example of the present application. As shown in FIG. 3, a medical imaging scanning system 300 includes a scanning apparatus 301 and a movable scan subject support apparatus 302. For example, the scan subject support apparatus 302 is connected to the scanning apparatus 301 by using a connection and fixation apparatus 303, so that the scan subject support apparatus 302 and the scanning apparatus 301 are fixed together.

In some examples, the connection and fixation apparatus 303 may be mounted on the side of the scanning apparatus 301 and/or the side of the scan subject support apparatus 302. The examples of the present application are not limited thereto. The connection and fixation apparatus 303 includes a fixation component. For details, refer to a related technology. For example, the connection and fixation apparatus includes a metal snap-fit structure, a resin snap-fit structure, or the like. In addition, the present application is not limited to a snap-fit or latching manner, for example, an adhesive manner or an electromagnetic engagement manner may be used.

The scan subject support apparatus 302 is fixed to the scanning apparatus 301 by using the connection and fixation apparatus 303, to prevent the movable scan subject support apparatus 302 from being separated from the scanning apparatus 301 due to collision or the like, that is, prevent the scan subject support apparatus 302 and the scanning apparatus 301 from being disconnected, thereby improving stability and safety during scanning. In addition, the connection and fixation apparatus 303 may accurately align the subject support apparatus 302 with the scanning apparatus 301, to accurately move an examination subject born on the subject support apparatus 302 to a scanning hole (refer to the scanning gantry opening 106 shown in FIG. 1) of the scanning apparatus 301 according to a scanning protocol, thereby improving scanning imaging quality.

In some examples, the medical imaging scanning system 300 may further include other components not shown. For details, refer to FIG. 1 and FIG. 2. In addition, for the scanning apparatus 301, also refer to FIG. 1 and FIG. 2 or refer to a related technology. The following further describes the scan subject support apparatus.

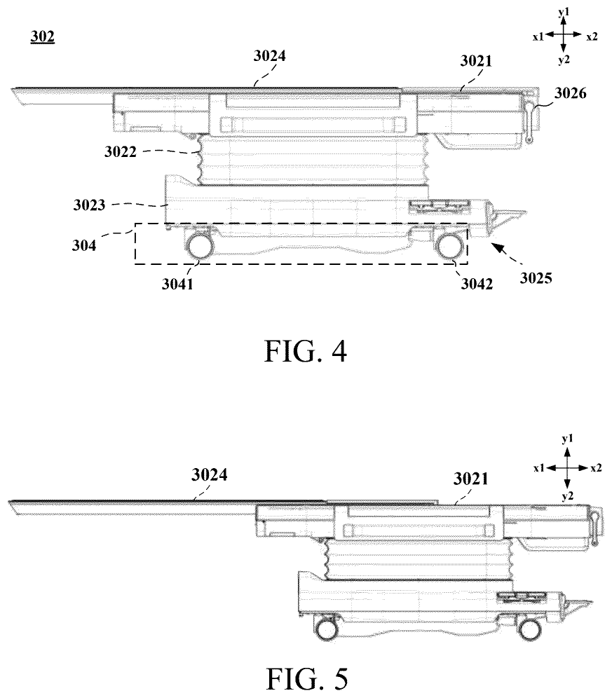

FIG. 4 is a schematic diagram of a scan subject support apparatus according to an example of the present application. As shown in FIG. 4, the scan subject support apparatus 302 includes a support platform 3021 for bearing a scan subject, a lifting drive portion 3022 for bearing the support platform 3021, a base 3023 for bearing the lifting drive portion 3022, a moving portion 304 mounted on the base 3023 and capable of moving the entire scan subject support apparatus 302, and an electromagnetic locking apparatus 3025 for fixing the scan subject support apparatus 302 (only some components are shown in FIG. 4).

The support platform 3021 is configured to bear the scan subject, the scan subject may lie on an upper surface of the support platform 3021, and a position of the scan subject relative to the scanning apparatus 301 is adjusted by moving the support platform 3021. The support platform 3021 has a movable support portion 3024, which can move in the length direction of the support platform 3021, that is, move in an x-axis direction (an x1 direction and an x2 direction shown in FIG. 4), to adjust a position of the scan subject in the x-axis direction, so that the scan subject is placed at a scanning position of the scanning apparatus 301. As shown in FIG. 4, the movable support portion 3024 is at an initial position. In this case, the scan subject lying on the movable support portion 3024 is within a range of the support platform 3021 in the up-down direction.

FIG. 5 is another schematic diagram of a scan subject support apparatus according to an example of the present application. As shown in FIG. 5, the movable support portion 3024 may move in the x1 direction, and at least a part thereof moves beyond the range of the support platform 3021 in the up-down direction, to move the scan subject to the scanning position. After scanning ends, the movable support portion 3024 may move in the x2 direction to return to the initial position.

In the examples of the present application, the support platform 3021 may also be referred to as a “scanning table”, a “medical bed”, a “patient table”, or the like, and the movable support portion 3024 of the support platform 3021 may also be referred to as a “bed plate”. This is not limited in the present application. The support platform 3021 is in a shape of a rectangular plate with a uniform thickness, and is configured to bear the scan subject, for example, the scan subject lies on the support platform 3021 in a supine posture.

In the examples of the present application, the movable support portion 3024 may also move the scan subject in the transverse direction of the support platform 3021 (that is, in the left-right direction, a direction perpendicular to an x axis and a y axis, or perpendicular to the drawing). The support platform 3021 may be made of a material having low X-ray attenuation, for example, made of a carbon fiber composite material. The examples of the present application are not limited thereto.

As shown in FIG. 4 and FIG. 5, the lifting drive portion 3022 is configured to bear the support platform 3021, and can adjust the vertical height of the support platform 3021 to adjust a position of the scan subject in the vertical direction, that is, a position in a y-axis direction (a y1 direction and a y2 direction shown in FIG. 4).

As shown in FIG. 4 and FIG. 5, the moving portion 304 is mounted on the base 3023 and can move the entire scan subject support apparatus 302. The moving portion 304 includes a plurality of casters disposed on the lower side of the base 3023. The plurality of casters include a first caster 3041 located on a side (that is, a side of the scan subject support apparatus 302 close to the scanning apparatus 301 during scanning) of a moving-out direction (the x1 direction shown in FIG. 4) in which the support platform 3021 moves toward the scanning apparatus 301 and a second caster 3042 located on a side (that is, a side of the scan subject support apparatus 302 away from the scanning apparatus 301 during scanning) opposite to a movement direction. That is, the first caster 3041 is located on the side of the x1 direction of the base 3023 or the side close to the scanning apparatus 301, and the second caster 3042 is located on the side of the x2 direction of the base 3023 or the side away from the scanning apparatus 301.

In this example of the present application, as shown in FIG. 5, after at least a part of the scan subject is removed from the support platform, the center of gravity of the entire movable scan subject support apparatus 302 changes. The center of gravity may deviate from the center of the scan subject support apparatus 302 and lean toward the first caster 3041. In this case, it is difficult to balance a load of the scan subject, causing the scan subject support apparatus 302 to tilt. In addition, for the moving portion 304 of the scan subject support apparatus 302 that includes the plurality of casters, the scan subject support apparatus 302 moves if the scan subject support apparatus 302 at a predetermined position is accidentally touched or impacted. In this case, re-positioning needs to be performed or quality of an image obtained through subsequent imaging scanning is affected. In this example of the present application, the electromagnetic locking apparatus may effectively maintain stability of the scan subject support apparatus and avoid accidental movement, thereby improving efficiency and imaging quality of an imaging scanning operation.

In this example of the present application, the electromagnetic locking apparatus 3025 includes a first anchoring portion disposed on the base 3023 and a second anchoring portion disposed at a position-to-be-fixed of the scan subject support apparatus 302. The electromagnetic locking apparatus 3025 engages the first anchoring portion with the second anchoring portion by using an electromagnetic force when being turned on, to fix the scan subject support apparatus 302 at the position-to-be-fixed. The following uses an example in which the position-to-be-fixed is located on the ground for description. The present application is not limited thereto. For example, the apparatus-to-be-fixed may alternatively be located on a wall or another place.

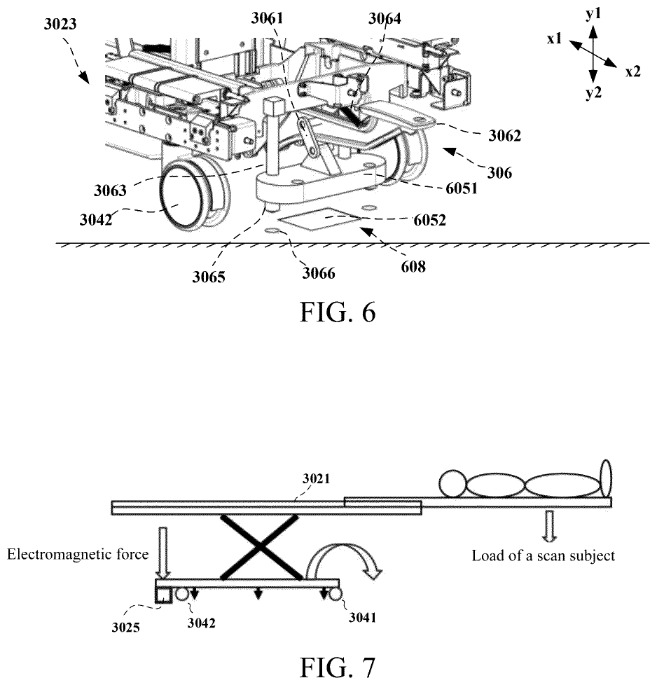

FIG. 6 is a schematic diagram of a local part of a scan subject support apparatus according to an example of the present application, and schematically shows some components of the electromagnetic locking apparatus 3025. As shown in FIG. 6, the first anchoring portion 6051 is disposed on a side of the base 3023 that faces the position-to-be-fixed 608 (that is, the lower side of the base 3023 that faces the ground), and is located at an end of the second caster 3042 (that is, an end away from the scanning apparatus 301). That is, the first anchoring portion 6051 is disposed on the side of the y2 direction (the lower side) of the base 3023 and is located on the side of the x2 direction (the right side) of the second caster 3042.

Therefore, the electromagnetic locking apparatus 3025 is disposed near the second caster 3042, so that when an anchoring fulcrum is the first caster 3041 after at least a part of the scan subject is removed from the support platform, a fixing moment can be further increased, thereby improving stability.

As shown in FIG. 6, the second anchoring portion 6052 is disposed on the ground and located at the position-to-be-fixed 608 of the scan subject support apparatus 302. The first anchoring portion 6051 and the second anchoring portion 6052 are engaged with each other by using the electromagnetic force generated when the electromagnetic locking apparatus 3025 is turned on, so that the scan subject support apparatus 302 is fixed on the ground.

In some examples, the first anchoring portion 6051 includes a magnetically conductive fixture (for example, a magnetically conductive steel plate), and the second anchoring portion 6052 disposed at the position-to-be-fixed 608 includes an electromagnet (for example, a magnetic lock). The electromagnetic force is generated by the magnetically conductive fixture and the electromagnet.

In some examples, the first anchoring portion 6051 includes an electromagnet (for example, a magnetic lock), and the second anchoring portion 6052 disposed at the position-to-be-fixed 608 includes a magnetically conductive fixture (for example, a magnetically conductive steel plate). The electromagnetic force is generated by the magnetically conductive fixture and the electromagnet.

For example, the magnetically conductive fixture may be made of a magnetically conductive material such as iron, cobalt, and/or nickel. The examples of the present application are not limited thereto. In addition, the electromagnet may use, for example, a related magnetic lock. For specific content or products such as the magnetic lock or electromagnetic engagement, refer to a related technology. This is not limited in the present application.

FIG. 7 is a schematic diagram of contact between a scan subject and a scan subject support platform according to an example of the present application. As shown in FIG. 7, the scan subject is located on the upper surface of the support platform 3021, and after the electromagnetic locking apparatus 3025 is turned on, the first anchoring portion 6051 is engaged with the second anchoring portion 6052 by using the electromagnetic force to fix the scan subject support apparatus 302 on the ground. Therefore, even if the scan subject on the support platform 3021 is moved out of the range of the base 3023, the first caster 3041 is a fulcrum of the scan subject support apparatus 302, and the electromagnetic locking apparatus 3025 can generate a sufficient electromagnetic force to balance the load of the scan subject, thereby maintaining stability of the scan subject support apparatus 302.

In the foregoing example, the moving portion 304 and the electromagnetic locking apparatus 3025 are disposed in the scan subject support apparatus 302, the scan subject is moved by the moving portion 304, and the first anchoring portion 6051 is engaged with the second anchoring portion 6052 by using the electromagnetic force after the electromagnetic locking apparatus 3025 is turned on, to fix the scan subject support apparatus 302 at the position-to-be-fixed.

When at least a part of the scan subject is removed from the support platform 3021, fixing the scan subject support apparatus 302 by using the electromagnetic force can maintain stability of the scan subject support apparatus 302, and prevent the scan subject support platform from tilting, thereby improving scanning safety. In addition, fixing the scan subject support apparatus 302 by using the electromagnetic force can avoid movement and further re-positioning that are caused when the scan subject support apparatus 302 is accidentally touched or impacted, or prevent quality of the image obtained through subsequent imaging scanning from being affected.

In some examples, the scan subject support apparatus 302 further includes a pressing apparatus 306. The pressing apparatus 306 is located on the lower side of the scan subject support apparatus 302, that is, on the side of the y2 direction. As shown in FIG. 6, the pressing apparatus 306 is connected to the first anchoring portion 6051 by using a connection module 3061, and can move the first anchoring portion 6051 toward the second anchoring portion 6052. Therefore, a distance of an electromagnetic mechanism is further shortened by using the pressing apparatus 306, thereby further increasing the electromagnetic force.

As shown in FIG. 6, the pressing apparatus 306 includes a pedal 3062 and a guide piece 3063. The pedal 3062 is mounted on the base 3023 and located on the side of the x2 direction of the base 3023. When the pedal 3062 is pressed, the first anchoring portion 6051 may move toward the second anchoring portion 6052 by using a rotating shaft. The guide piece 3063 guides the first anchoring portion 6051, limits a direction in which the first anchoring portion 6051 moves toward the second anchoring portion 6052, and limits a direction in which the first anchoring portion 6051 moves away from the second anchoring portion 6052.

For example, the guide piece 3063 is two posts shown in FIG. 6. The guide piece 3063 limits the first anchoring portion 6051 to move toward the second anchoring portion 6052 in the y2 direction (downward movement), and limits the first anchoring portion 6051 to move away from the second anchoring portion 1052 in the y1 direction (upward movement). The examples of the present application are not limited thereto.

Therefore, the support apparatus can be fixed by using simple structures such as the pedal 3062 and the guide piece 3063, and a distance between the first anchoring portion and the second anchoring portion is shortened to increase the electromagnetic force, thereby further improving operation convenience.

In some examples, the pressing apparatus 306 further includes a restoration piece 3064. When the electromagnetic locking apparatus 3025 is turned off, the pressing apparatus 306 in a pressed state is restored to an initial position. In this case, the first anchoring portion 6051 is away from the second anchoring portion 6052.

As shown in FIG. 6, for example, the restoration piece 3064 may include a spring. The pressing apparatus 306 is restored to a non-pressed state by using an elastic force of the spring when the electromagnetic locking apparatus 3025 is turned off, that is, the pedal 3062 is restored to an initial position. FIG. 6 is described by using an example in which the restoration piece 3064 includes a spring, but the restoration piece may alternatively be another elastic restoration apparatus. The examples of the present application are not limited thereto. Therefore, an initial state is automatically restored by using the restoration piece 3064 after scanning, thereby improving operation convenience.

In some examples, the pressing apparatus 306 further includes a positioning piece 3065 protruding downward from the first anchoring portion 6051. As shown in FIG. 6, both the second anchoring portion 6052 and a positioning hole 3066 are disposed at the position-to-be-fixed 608.

For example, if a position of the positioning piece 3065 corresponds to a position of the positioning hole 3066 in the up-down direction, when the pressing apparatus 306 is pressed to move the first anchoring portion 6051 toward the second anchoring portion 6052, the positioning piece 3065 is inserted into the positioning hole 3066. In this case, the first anchoring portion 6051 is engaged with the second anchoring portion 6052 by using the electromagnetic force, so that the scan subject support apparatus 302 is fixed at the position-to-be-fixed 608. When the pressing apparatus 306 is restored to the non-pressed state, the positioning piece 3065 is separated from the positioning hole 3066.

For another example, if a position of the positioning piece 3065 does not correspond to a position of the positioning hole 3066 in the up-down direction, when the pressing apparatus 306 is pressed to move the first anchoring portion 6051 toward the second anchoring portion 6052, the positioning piece 3065 cannot be inserted into the positioning hole 3066. In this case, no sufficient engagement force is generated between the first anchoring portion 6051 and the second anchoring portion 6052, and a position of the scan subject support apparatus 302 needs to be adjusted. Therefore, the positioning piece 3065 and the positioning hole 3066 can further improve operation accuracy, and avoid a case that scanning starts before fixing of the scan subject support apparatus 302 completes, thereby further improving safety.

In some examples, the scan subject support apparatus 302 further includes a handle portion 3026. As shown in FIG. 4, for example, the handle portion 3026 is located on a side opposite to the moving-out direction in which the support platform 3021 moves toward the scanning apparatus 301, that is, located on the side of the x2 direction or a side of the scan subject support apparatus 302 away from the scanning apparatus 301 during scanning, to control the scan subject support apparatus 302 to move. The handle portion 3026 includes a magnetic switch, which turns on or off the electromagnetic locking apparatus by turning on or off a power supply.

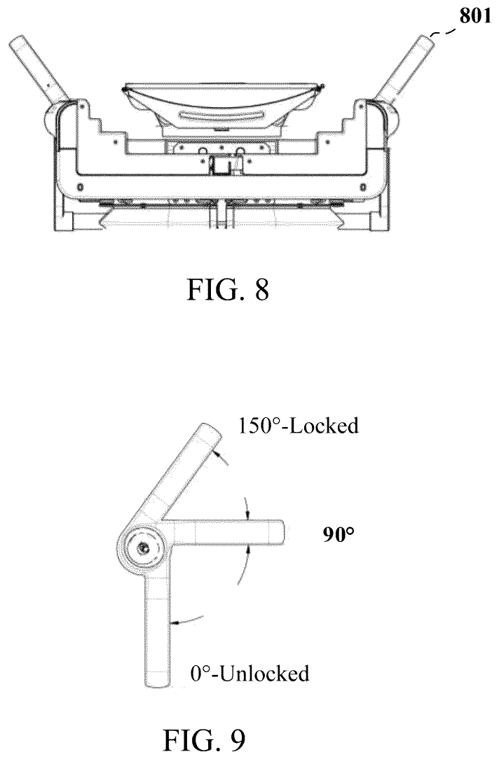

FIG. 8 is a schematic diagram of a handle portion according to an example of the present application, and shows a partial case of the scan subject support apparatus 302 viewed from an x2 side. As shown in FIG. 8, for example, the handle portion 3026 may include a magnetic switch 801. Therefore, the handle portion 3026 may control the scan subject support apparatus 302 to move, to improve convenience of a movement operation, and may also control on/off of the electromagnetic locking apparatus 3025, to improve convenience of an electromagnetic fixation operation.

FIG. 9 is a schematic diagram of a magnetic switch according to an example of the present application, and schematically shows cases that the magnetic switch 801 is located at different positions. As shown in FIG. 9, an angle of the magnetic switch 801 may be adjusted according to a requirement of a user, for example, 0°, 90°, or 150°. The examples of the present application are not limited thereto.

For example, the magnetic switch 801 may be adjusted to a specific angle to turn on the power supply, to turn on the electromagnetic locking apparatus 3025. For example, when the angle of the magnetic switch 801 is adjusted to 150°, the electromagnetic locking apparatus is turned on (that is, “Locked” shown in FIG. 9); and when the angle of the magnetic switch is adjusted to 0°, the electromagnetic locking apparatus is turned off (that is, “Unlocked” shown in FIG. 9).

For another example, a member of medical staff may adjust the angle of the magnetic switch 801 to 90° to move the scan subject lying on the scan subject support apparatus to near a CT device, thereby facilitating movement control of the scan subject support apparatus. After the scan subject support apparatus is connected to the CT device by using the connection and fixation apparatus, the medical staff adjusts the angle to 150°. In this case, the power supply is turned on, and the electromagnetic locking apparatus is turned on. After scanning is completed, when the medical staff adjusts the angle to any angle other than 150°, the power supply is turned off and the electromagnetic locking apparatus is turned off.

FIG. 8 and FIG. 9 are described by using an example in which the handle portion 3026 is the magnetic switch 801. The present application is not limited thereto. For example, the magnetic switch may alternatively be one or a plurality of buttons located on the handle portion 3026, and the button is pressed to turn on/off the power supply to turn on/off the electromagnetic locking apparatus.

The above examples merely provide illustrative descriptions of the examples of the present application. However, the present application is not limited thereto, and suitable variations may be made on the basis of the above examples. For example, each of the above examples may be used independently, or one or more of the above examples may be combined.

An example of the present application further provides an anchoring control method, applied to a movable scan subject support apparatus. The scan subject support apparatus includes a support platform for bearing a scan subject, a lifting drive portion for bearing the support platform, a base for bearing the lifting drive portion, a moving portion, and an electromagnetic locking apparatus.

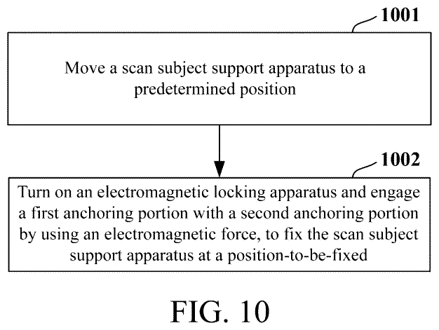

FIG. 10 is a schematic diagram of an anchoring control method according to an example of the present application. As shown in FIG. 10, the method includes moving a scan subject support apparatus to a predetermined position (1001). The electromagnetic locking apparatus includes a first anchoring portion disposed on the base and a second anchoring portion disposed at a position-to-be-fixed of the scan subject support apparatus.

The method further includes turning on an electromagnetic locking apparatus and engaging a first anchoring portion with a second anchoring portion by using an electromagnetic force, to fix the scan subject support apparatus at a position-to-be-fixed (1002).

It should be noted that FIG. 10 merely schematically illustrates an example of the present application. The present application is not limited thereto. For example, some of the above steps may be executed simultaneously, or may be executed in a sequential order. The order of execution between operations may be appropriately adjusted. In addition, some other operations may be added or some operations may be omitted. A person skilled in the art may make appropriate variations based on the described content, rather than being limited to what is set forth in FIG. 10.

In this example of the present application, after the electromagnetic locking apparatus is turned on, the first anchoring portion is engaged with the second anchoring portion by using the electromagnetic force, so that the movable scan subject support apparatus is fixed at the position-to-be-fixed. Therefore, a scanning operation step can be simplified, tension of a scan subject is relieved, stability of the scan subject support apparatus is maintained, and problems that scanning cannot be completed or even the scan subject support platform collides with the scanning apparatus caused by tilting of the scan subject support platform are avoided.

In some examples, after the scan subject support apparatus is moved to the predetermined position, the scan subject support apparatus is fixed to the scanning apparatus by using a connection and fixation apparatus. The connection and fixation apparatus is located on the side of the scanning apparatus, and/or the connection and fixation apparatus is located on the side of the scan subject support apparatus. The examples of the present application are not limited thereto. The connection and fixation apparatus includes a fixation structure such as a latching structure or a locking structure. The examples of the present application are not limited thereto.

For example, a medical staff moves the scan subject support apparatus to near the scanning apparatus, and the scan subject support apparatus is fixed by using the fixation structure, to prevent the scanning apparatus and the scan subject support apparatus from being disconnected. After scanning is completed, the medical staff opens the fixation structure. In this case, the scan subject support apparatus can move.

Therefore, the scanning apparatus and the scan subject support apparatus can be prevented from being disconnected by collision or the like.

In some examples, a power supply is turned on by using a magnetic switch of the scan subject support apparatus, to turn on the electromagnetic locking apparatus. For example, the magnetic switch is located on a handle portion, and the power supply is turned on by adjusting the handle portion to a specific angle, for example, adjusting an angle of the handle portion to 150°, to turn on the electromagnetic locking apparatus. The magnetic switch may alternatively be a button located on the handle portion, and the button is pressed to turn on the power supply to turn on the electromagnetic locking apparatus. The examples of the present application are not limited thereto.

For example, after the scan subject support apparatus is connected to a CT device by using the connection and fixation apparatus, the medical staff adjusts the angle of the handle portion to a maximum angle of 150°. In this case, the power supply is turned on, the electromagnetic locking apparatus is turned on, and the first anchoring portion is engaged with the second anchoring portion by using the electromagnetic force, so that the scan subject support apparatus is fixed at the predetermined position. After scanning is completed, the medical staff adjusts the angle of the handle portion to any angle other than 150°. In this case, the power supply is turned off, the electromagnetic locking apparatus is turned off, and the first anchoring portion and the second anchoring portion are separated, so that the medical staff can move the scan subject support apparatus to another position.

In some examples, after the electromagnetic locking apparatus is turned on, a distance between the first anchoring portion and the second scanning portion is shortened by using a pressing apparatus of the scan subject support apparatus. The first anchoring portion is guided by using a guide piece, and the first anchoring portion is positioned by using a positioning piece and a positioning hole.

For example, after the electromagnetic locking apparatus is turned on, the pressing apparatus of the scan subject support apparatus is pressed by stepping on a pedal. In this case, the first anchoring portion moves toward the second anchoring portion based on the guide piece. If positions of the positioning piece and the positioning hole are inconsistent, the positioning piece protruding downward from the first anchoring portion on the pressing apparatus contacts the ground, that is, the positioning piece cannot be inserted into the positioning hole, and the first anchoring portion and the second anchoring portion cannot be completely engaged with each other. Therefore, it is difficult to maintain stability of the scan subject support apparatus. If positions of the positioning piece and the positioning hole are consistent, the positioning piece is inserted into the positioning hole, and the first anchoring portion and the second anchoring portion are engaged by using the electromagnetic force, so that the scan subject support apparatus is fixed at a fixed position on the ground.

Therefore, it can be ensured that after the electromagnetic locking apparatus is turned on, the first anchoring portion and the second anchoring portion that are engaged can fix the scan subject support apparatus to the ground, thereby maintaining stability of the scan subject support apparatus. In addition, a position of the scan subject support apparatus can be further determined, to prevent a movable support portion moved out from touching the scanning apparatus due to a position deviation.

In some examples, the power supply is turned off by using the magnetic switch of the scan subject support apparatus, to turn off the electromagnetic locking apparatus. In addition, the pressing apparatus is restored to an initial position by using a restoration piece of the pressing apparatus.

For example, the medical staff adjusts the angle of the handle portion to any angle other than 150°. In this case, the magnetic switch is turned off, the power supply is turned off, the electromagnetic locking apparatus is turned off, and there is no electromagnetic force between the first anchoring portion and the second anchoring portion. The pressing apparatus automatically restores to a non-pressed state under the action of the restoration piece.

Therefore, an operation procedure can be simplified, and there is no need to separate the first anchoring portion from the second anchoring portion manually.

The above examples merely provide illustrative descriptions of the examples of the present application. However, the present application is not limited thereto, and suitable variations may be made on the basis of the above examples. For example, each of the above examples may be used independently, or one or more of the above examples may be combined.

The present application is described above with reference to specific implementations. However, it should be clear to those skilled in the art that the foregoing description is merely illustrative and is not intended to limit the scope of protection of the present application. Various variations and modifications may be made by those skilled in the art according to the spirit and principle of the present application, and these variations and modifications also fall within the scope of the present application.

Preferred examples of the present application are described above with reference to the accompanying drawings. Many features and advantages of the examples are clear according to the detailed description. Therefore, the appended claims are intended to cover all these features and advantages that fall within the true spirit and scope of these examples. In addition, as many modifications and changes could be easily conceived of by those skilled in the art, the examples of the present application are not limited to the illustrated and described precise structures and operations, but can encompass all appropriate modifications, changes, and equivalents that fall within the scope of the examples.

1. A scan subject support apparatus, comprising:
a support platform for bearing a scan subject,
a lifting drive portion for bearing the support platform; and
a base for bearing the lifting drive portion, wherein the base includes:
a moving portion, mounted on the base and capable of moving the entire scan subject support apparatus; and
an electromagnetic locking apparatus, including a first anchoring portion disposed on the base and a second anchoring portion disposed at a position-to-be-fixed of the scan subject support apparatus, wherein the electromagnetic locking apparatus engages the first anchoring portion with the second anchoring portion by using an electromagnetic force when being turned on, to fix the scan subject support apparatus at the position-to-be-fixed.

2. The scan subject support apparatus according to claim 1, wherein, the moving portion includes a plurality of casters disposed on a lower side of the base, the plurality of casters including a first caster located on a side of the scan subject support apparatus close to a scanning apparatus during scanning and a second caster located on a side away from the scanning apparatus, and wherein the first anchoring portion is disposed at an end of the base away from the scanning apparatus, and is located on a same side of the base as the second caster.

3. The scan subject support apparatus according to claim 1, wherein, the first anchoring portion includes a magnetically conductive fixture, the second anchoring portion disposed at the position-to-be-fixed includes an electromagnet, and the electromagnetic force is generated by the magnetically conductive fixture and the electromagnet, or wherein the first anchoring portion includes an electromagnet, the second anchoring portion disposed at the position-to-be-fixed includes a magnetically conductive fixture, and the electromagnetic force is generated by the magnetically conductive fixture and the electromagnet.

4. The scan subject support apparatus according to claim 1, further including a pressing apparatus located on a lower side of the scan subject support apparatus and connected to the first anchoring portion by using a connection module, and capable of moving the first anchoring portion toward the second anchoring portion.

5. The scan subject support apparatus according to claim 4, wherein the pressing apparatus includes:
a pedal, moving the first anchoring portion toward the second anchoring portion by using a rotating shaft; and
a guide piece, guiding the first anchoring portion, to limit a direction in which the first anchoring portion moves toward the second anchoring portion.

6. The scan subject support apparatus according to claim 5, wherein the pressing apparatus further includes a restoration piece, used to restore the pressing apparatus in a pressed state to an initial position when the electromagnetic locking apparatus is turned off.

7. The scan subject support apparatus according to claim 5, wherein the second anchoring portion is disposed on the ground, and a positioning hole is further disposed at the position-to-be-fixed, wherein the pressing apparatus further includes a positioning piece protruding downward from the first anchoring portion, and the positioning piece is inserted into the positioning hole when the pressing apparatus is pressed to move the first anchoring portion toward the second anchoring portion, and the positioning piece is separated from the positioning hole when the pressing apparatus is restored to a non-pressed state.

8. The scan subject support apparatus according to claim 1, further including a handle portion, located on a side of the scan subject support apparatus away from a scanning apparatus during scanning, and capable of controlling the scan subject support apparatus to move.

9. The scan subject support apparatus according to claim 8, wherein the handle portion includes a magnetic switch, turning on or off the electromagnetic locking apparatus by turning on or off a power supply.

10. The scan subject support apparatus according to claim 1, further including a connection and fixation apparatus, capable of fixing the scan subject support apparatus to a scanning apparatus.

11. An anchoring control method, applied to a movable scan subject support apparatus, wherein the scan subject support apparatus includes a support platform for bearing a scan subject, a lifting drive portion for bearing the support platform, and a base for bearing the lifting drive portion, the scan subject support apparatus further includes a moving portion and an electromagnetic locking apparatus, and the method comprises:
moving the scan subject support apparatus to a predetermined position, wherein the electromagnetic locking apparatus includes a first anchoring portion disposed on the base and a second anchoring portion disposed at a position-to-be-fixed of the scan subject support apparatus; and
turning on the electromagnetic locking apparatus and engaging the first anchoring portion with the second anchoring portion by using an electromagnetic force, to fix the scan subject support apparatus at the position-to-be-fixed.

12. The method according to claim 11, after the moving the scan subject support apparatus to a predetermined position, further including fixing, by using a connection and fixation apparatus, the scan subject support apparatus to a scanning apparatus.

13. The method according to claim 11, further including turning on, by using a magnetic switch of the scan subject support apparatus, a power supply to turn on the electromagnetic locking apparatus.

14. The method according to claim 14, further including shortening, by using a pressing apparatus of the scan subject support apparatus, a distance between the first anchoring portion and the second anchoring portion, wherein the first anchoring portion is guided by using a guide piece, and the first anchoring portion is positioned by using a positioning piece and a positioning hole.

15. The method according to claim 14, further including:
turning off, by using the magnetic switch of the scan subject support apparatus, the power supply to turn off the electromagnetic locking apparatus; and
restoring, by using a restoration piece of the pressing apparatus, the pressing apparatus to an initial position.
